(19) United States
(12) Patent Application Publication
(10) Pub. No.: US 2019/0388156 A1
(43) Pub. Date: Dec. 26, 2019

(54) MEDICAL INSTRUMENT WITH MULTI-COIL POSITION SENSOR

(71) Applicant: Acclarent, Inc., Irvine, CA (US)

(72) Inventor: Ehsan Shameli, Irvine, CA (US)

(21) Appl. No.: 16/012,841

(22) Filed: Jun. 20, 2018

(51) Int. Cl. A61B 34/20; A61M 29/02

(52) U.S. Cl. CPC A61B 34/20; A61M 29/02; A61M 2210/0681; A61B 2034/2055; A61B 2034/2051; A61B 2034/2057; A61M 2210/0675

(57) ABSTRACT

An apparatus includes a medical instrument, a sensor assembly, and a pair of wires. The sensor assembly is positioned at the distal end of the medical instrument. The sensor assembly includes a first coil member oriented along a first longitudinal axis and a second coil member oriented along a second longitudinal axis. The first and second coil members are coupled with each other in series. The first and second longitudinal axes are offset from each other. The first and second coil members are configured to generate electrical signals indicative of a position and orientation of the distal end of the medical instrument within a patient. First and second wires of the pair of wires are operable to communicate signals from the sensor assembly to a navigation system.

### BACKGROUND

In some instances, it may be desirable to dilate an anatomical passageway in a patient. This may include dilation of ostia of paranasal sinuses (e.g., to treat sinusitis), dilation of the larynx, dilation of the Eustachian tube, dilation of other passageways within the ear, nose, or throat, etc. One method of dilating anatomical passageways includes using a guide wire and catheter to position an inflatable balloon within the anatomical passageway, then inflating the balloon with a fluid (e.g., saline) to dilate the anatomical passageway. For instance, the expandable balloon may be positioned within an ostium at a paranasal sinus and then be inflated, to thereby dilate the ostium by remodeling the bone adjacent to the ostium, without requiring incision of the mucosa or removal of any bone. The dilated ostium may then allow for improved drainage from and ventilation of the affected paranasal sinus. A system that may be used to perform such procedures may be provided in accordance with the teachings of U.S. Pub. No. 2011/0004057, entitled “Systems and Methods for Transnasal Dilation of Passageways in the Ear, Nose or Throat,” published Jan. 6, 2011, the disclosure of which is incorporated by reference herein. An example of such a system is the Relieva® Spin Balloon Sinuplasty™ System by Acclarent, Inc. of Irvine, Calif.

Image-guided surgery (IGS) is a technique where a computer is used to obtain a real-time correlation of the location of an instrument that has been inserted into a patient's body to a set of preoperatively obtained images (e.g., a CT or MRI scan, 3-D map, etc.), such that the computer system may superimpose the current location of the instrument on the preoperatively obtained images. In some IGS procedures, a digital tomographic scan (e.g., CT or MRI, 3-D map, etc.) of the operative field is obtained prior to surgery. A specially programmed computer is then used to convert the digital tomographic scan data into a digital map. During surgery, special instruments having sensors (e.g., electromagnetic coils that emit electromagnetic fields and/or are responsive to externally generated electromagnetic fields) mounted thereon are used to perform the procedure while the sensors send data to the computer indicating the current position of each surgical instrument. The computer correlates the data it receives from the instrument-mounted sensors with the digital map that was created from the preoperative tomographic scan. The tomographic scan images are displayed on a video monitor along with an indicator (e.g., crosshairs or an illuminated dot, etc.) showing the real-time position of each surgical instrument relative to the anatomical structures shown in the scan images. In this manner, the surgeon is able to know the precise position of each sensor-equipped instrument by viewing the video monitor even if the surgeon is unable to directly visualize the instrument itself at its current location within the body.

An example of an electromagnetic IGS systems that may be used in ENT and sinus surgery is the CARTO® 3 System by Biosense-Webster, Inc., of Irvine, Calif. When applied to functional endoscopic sinus surgery (FESS), balloon sinuplasty, and/or other ENT procedures, the use of IGS systems allows the surgeon to achieve more precise movement and positioning of the surgical instruments than can be achieved by viewing through an endoscope alone. As a result, IGS systems may be particularly useful during performance of FESS, balloon sinuplasty, and/or other ENT procedures where anatomical landmarks are not present or are difficult to visualize endoscopically.

Navigation of the three-dimensional views of the areas surrounding the operative field (e.g., rotating or moving a viewpoint within three-dimensional space) may be accomplished via interaction with an interface device, such as a keyboard or mouse, of an IGS system. These types of interface devices might not be intended for use in a sterile environment, and therefore may not located within reach of a clinician that is performing a medical procedure with the assistance of an IGS system. As a result, clinicians may need to relay navigation instructions to an assistant in another room or area, who will then use the interface device to provide the three-dimensional views that the clinician desires. This process can be time consuming and error prone.

While several systems and methods have been made and used in ENT procedures, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1A depicts a perspective view of an exemplary dilation instrument assembly, with a guidewire in a proximal position, and with a dilation catheter in a proximal position;

FIG. 1B depicts a perspective view of the dilation instrument assembly of FIG. 1A, with the guidewire in a distal position, and with the dilation catheter in the proximal position;

FIG. 1C depicts a perspective view of the dilation instrument assembly of FIG. 1A, with the guidewire in a distal position, with the dilation catheter in a distal position, and with a dilator of the dilation catheter in a non-dilated state;

FIG. 1D depicts a perspective view of the dilation instrument assembly of FIG. 1A, with the guidewire in a distal position, with the dilation catheter in the distal position, and with a dilator of the dilation catheter in a dilated state;

FIG. 2 depicts a schematic view of an exemplary sinus surgery navigation system being used on a patient seated in an exemplary medical procedure chair;

FIG. 3 depicts a side schematic view of a distal end portion of a navigation guidewire of the system of FIG. 2;

FIG. 4 depicts a side elevational view of an exemplary sensor coil that may be incorporated into the navigation guidewire of FIG. 3;

FIG. 5 depicts a perspective view of an exemplary alternative sensor coil assembly that may be incorporated into the navigation guidewire of FIG. 3;

FIG. 6 depicts a schematic perspective view of an exemplary alternative medical instrument that may be used with the system of FIG. 2, including a pair of sensors connected in series;

FIG. 7 depicts a schematic perspective view of another exemplary alternative medical instrument that may be used with the system of FIG. 2, including a pair of sensors connected in series; and

FIG. 8 depicts a schematic perspective view of another exemplary alternative medical instrument that may be used with the system of FIG. 2, including a pair of sensors connected in series.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

It will be appreciated that the terms “proximal” and “distal” are used herein with reference to a clinician gripping a handpiece assembly. Thus, an end effector is distal with respect to the more proximal handpiece assembly. It will be further appreciated that, for convenience and clarity, spatial terms such as “top” and “bottom” also are used herein with respect to the clinician gripping the handpiece assembly. However, surgical instruments are used in many orientations and positions, and these terms are not intended to be limiting and absolute.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

### I. EXEMPLARY DILATION CATHETER SYSTEM

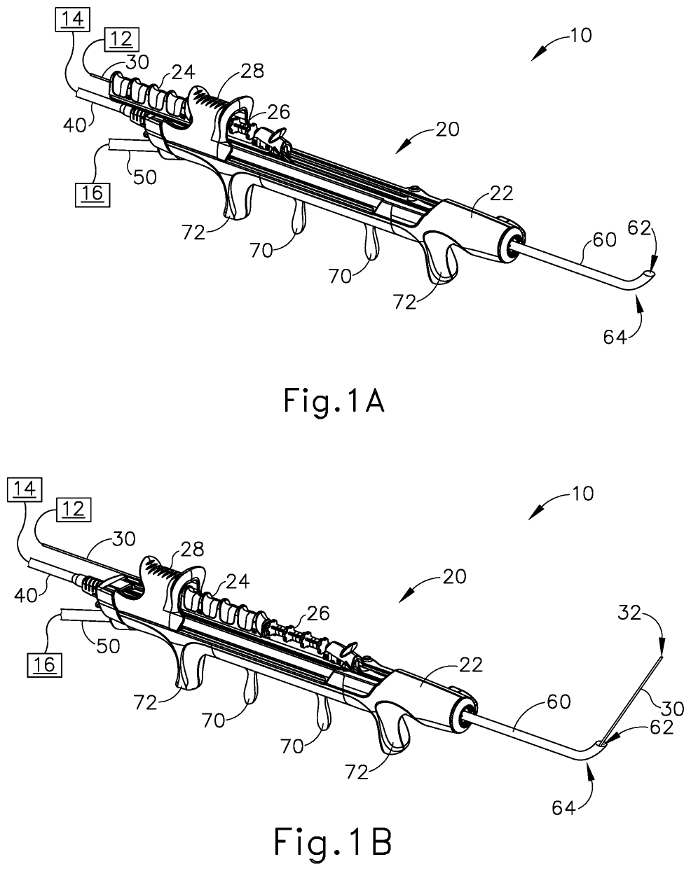

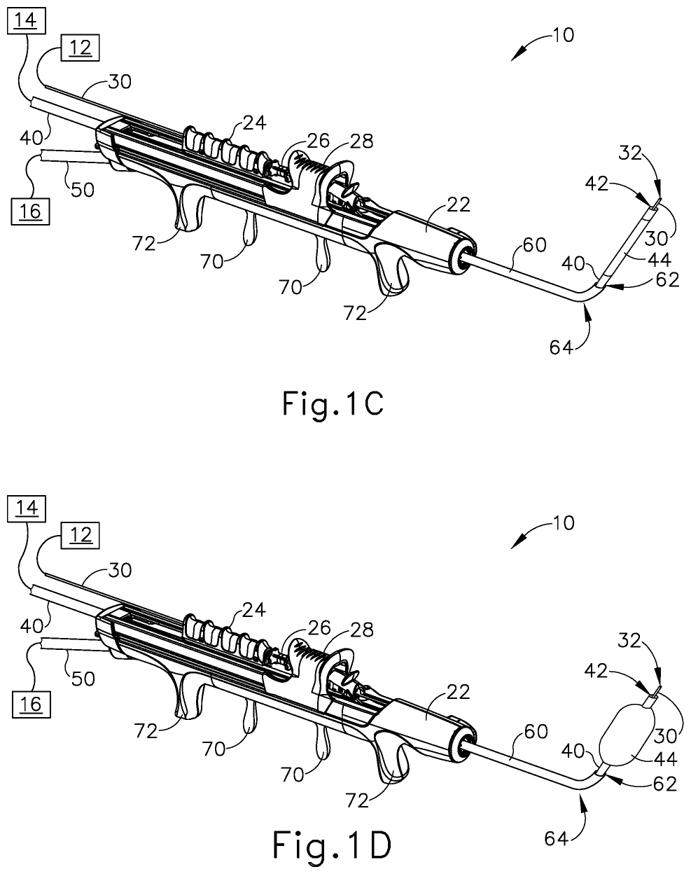

FIGS. 1A-ID show an exemplary dilation instrument assembly (10) that may be used to dilate the ostium of a paranasal sinus; to dilate some other passageway associated with drainage of a paranasal sinus; to dilate a Eustachian tube; or to dilate some other anatomical passageway (e.g., within the ear, nose, or throat, etc.). Dilation instrument assembly (10) of this example comprises a guidewire power source (12), an inflation source (14), an irrigation fluid source (16), and a dilation instrument (20). In some versions, guidewire power source (12) comprises a source of light. In some other versions, guidewire power source (12) is part of an IGS system as described below. Inflation source (14) may comprise a source of saline or any other suitable source of fluid. Irrigation fluid source (16) may comprise a source of saline or any other suitable source of fluid. Irrigation fluid source (16) may be omitted in some versions.

Dilation instrument (20) of the present example comprise a handle body (22) with a guidewire slider (24), a guidewire spinner (26), and a dilation catheter slider (28). Handle body (22) is sized and configured to be gripped by a single hand of a human operator. Sliders (24, 28) and spinner (26) are also positioned and configured to be manipulated by the same hand that grasps handle body (22).

A guide catheter (60) extends distally from handle body (22). Guide catheter (60) includes an open distal end (62) and a bend (64) formed proximal to open distal end (62). Dilation instrument (20) is configured to removably receive several different kinds of guide catheters (60), each guide catheter (60) having a different angle formed by bend (64). Guide catheter (60) of the present example is formed of a rigid material, such that guide catheter (60) maintains a consistent configuration of bend (64) during use of dilation instrument (20). In some versions, dilation instrument (20), is further configured to enable rotation of guide catheter (60), relative to handle body (22), about the longitudinal axis of the straight proximal portion of guide catheter (60).

A guidewire (30) is coaxially disposed in guide catheter (60). Guidewire slider (24) is secured to guidewire (30). Translation of guidewire slider (24) relative to handle body (22) from a proximal position (FIG. 1A) to a distal position (FIG. 1B) causes corresponding translation of guidewire (30) relative to handle body (22) from a proximal position (FIG. 1A) to a distal position (FIG. 1B). When guidewire (30) is in a distal position, a distal portion of guidewire (30) protrudes distally from open distal end (62) of guide catheter (60). Guidewire spinner (26) is operable to rotate guidewire (30) about the longitudinal axis of guidewire (30). Guidewire spinner (26) is coupled with guidewire slider (24) such that guidewire spinner (26) translates longitudinally with guidewire slider (24). By way of example only, guidewire (30) may be configured in accordance with at least some of the teachings of U.S. Pat. No. 9,155,492, the disclosure of which is incorporated by reference herein. Other features and operabilities that may be incorporated into guidewire (30) will be apparent to those skilled in the art in view of the teachings herein.

A dilation catheter (40) is coaxially disposed in guide catheter (60). Dilation catheter slider (28) is secured to dilation catheter (40). Translation of dilation catheter slider (28) relative to handle body (22) from a proximal position (FIG. 1B) to a distal position (FIG. 1C) causes corresponding translation of dilation catheter (40) relative to handle body (22) from a proximal position (FIG. 1B) to a distal position (FIG. 1C). When dilation catheter (40) is in a distal position, a distal portion of dilation catheter (40) protrudes distally from open distal end (62) of guide catheter (60). Dilation catheter (40) of the present example comprises a non-extensible balloon (44) located just proximal to open distal end (42) of dilation catheter (40). Balloon (44) is in fluid communication with inflation source (14). Inflation source (14) is configured to communicate fluid (e.g., saline, etc.) to and from balloon (44) to thereby transition balloon (44) between a non-inflated state and an inflated state. FIG. 1C shows balloon (44) in a non-inflated state. FIG. 1D shows balloon (44) in an inflated state. In the non-inflated state, balloon (44) is configured to be inserted into a constricted anatomical passageway. In the inflated state, balloon (44) is configured to dilate the anatomical passageway in which balloon (44) is inserted. Other features and operabilities that may be incorporated into dilation catheter (40) will be apparent to those skilled in the art in view of the teachings herein.

### II. EXEMPLARY IMAGE GUIDED SURGERY NAVIGATION SYSTEM

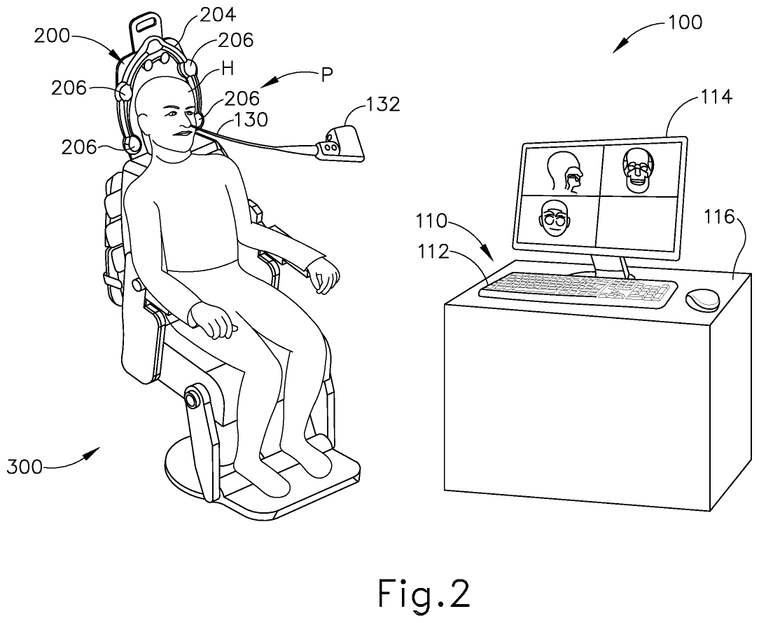

FIG. 2 shows an exemplary IGS navigation system (100) enabling an ENT procedure to be performed using image guidance. In some instances, IGS navigation system (100) is used during a procedure where dilation instrument assembly (10) is used to dilate the ostium of a paranasal sinus; or to dilate some other anatomical passageway (e.g., within the ear, nose, or throat, etc.). Navigation guidewire (130) of IGS navigation system (100) may be used as a substitute for guidewire (30) in dilation instrument (20) described above. In addition to or in lieu of having the components and operability described herein IGS navigation system (100) may be constructed and operable in accordance with at least some of the teachings of U.S. Pat. No. 7,720,521, entitled “Methods and Devices for Performing Procedures within the Ear, Nose, Throat and Paranasal Sinuses,” issued May 18, 2010, the disclosure of which is incorporated by reference herein; U.S. Pat. Pub. No. 2016/0310042, entitled “System and Method to Map Structures of Nasal Cavity,” published Oct. 27, 2016, the disclosure of which is incorporated by reference herein; U.S. Pat. Pub. No. 2014/0364725, entitled “Systems and Methods for Performing Image Guided Procedures within the Ear, Nose, Throat and Paranasal Sinuses,” published Dec. 11, 2014, the disclosure of which is incorporated by reference herein; and U.S. patent application Ser. No. 15/933,737, entitled “Apparatus to Secure Field Generating Device to Chair,” filed Mar. 23, 2018, the disclosure of which is incorporated by reference herein.

IGS navigation system (100) of the present example comprises a field generator assembly (200), which comprises set of magnetic field generators (206) that are integrated into a horseshoe-shaped frame (204). Field generators (206) are operable to generate alternating magnetic fields of different frequencies around the head of the patient. In the present example, frame (204) is mounted to a chair (300), with the patient (P) being seated in the chair (300) such that frame (204) is located adjacent to the head (H) of the patient (P). IGS navigation system (100) of the present example further comprises a processor (110), which controls field generators (206) and other elements of IGS navigation system (100) as described below. Processor (110) of the present example is mounted in a console (116), which comprises operating controls (112) that include a keypad and/or a pointing device such as a mouse or trackball. A physician uses operating controls (112) to interact with processor (110) while performing the surgical procedure.

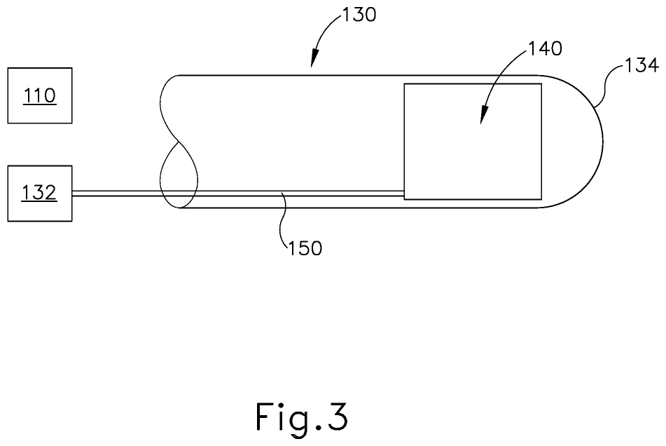

A coupling unit (132) is secured to the proximal end of a navigation guidewire (130). Coupling unit (132) of this example is configured to provide wireless communication of data and other signals between console (116) and navigation guidewire (130). Other versions may provide wired coupling between coupling unit (132) and console (116). As shown in FIG. 3, navigation guidewire (130) includes a sensor (140) that is located at distal end (134) of navigation guidewire (130). Sensor (140) is in communication with coupling unit (132) via one or more wires (150) extending along the length of navigation guidewire (130). Sensor (140) is responsive to positioning within the alternating electromagnetic generated by field generators (206). In the present example, sensor (140) comprises at least one coil at distal end (134) of navigation guidewire (130). When such a coil is positioned within an alternating current electromagnetic field generated by field generators (206), the alternating magnetic field may generate electrical current in the coil, and this electrical current may be communicated along the electrical conduit(s) in navigation guidewire (130) and further to processor (110) via coupling unit (132). This phenomenon may enable IGS navigation system (100) to determine the location of the distal end (134) of navigation guidewire (130) within a three-dimensional space (i.e., within the head (H) of the patient (P)). To accomplish this, processor (110) executes an algorithm to calculate location coordinates of the distal end (134) of navigation guidewire (130) from the position related signals of the coil(s) in sensor (140) of navigation guidewire (130).

Processor (110) uses software stored in a memory of processor (110) to drive field generators (206), process data from navigation guidewire (130), process data from operating controls (112), and drive display screen (114). Processor (110) is further operable to provide video in real time via display screen (114), showing the position of the distal end of navigation guidewire (130) in relation to a video camera image of the patient's head (H), a CT scan image of the patient's head (H), and/or a computer generated three-dimensional model of the anatomy within and adjacent to the patient's nasal cavity. Display screen (114) may display such images simultaneously and/or superimposed on each other during the surgical procedure. Such displayed images may also include graphical representations of instruments that are inserted in the patient's head (H), such as navigation guidewire (130), such that the operator may view the virtual rendering of the instrument at its actual location in real time. The images provided through display screen (114) may help guide the operator in maneuvering and otherwise manipulating instruments within the patient's head (H).

### III. EXEMPLARY SENSOR COIL CONFIGURATIONS

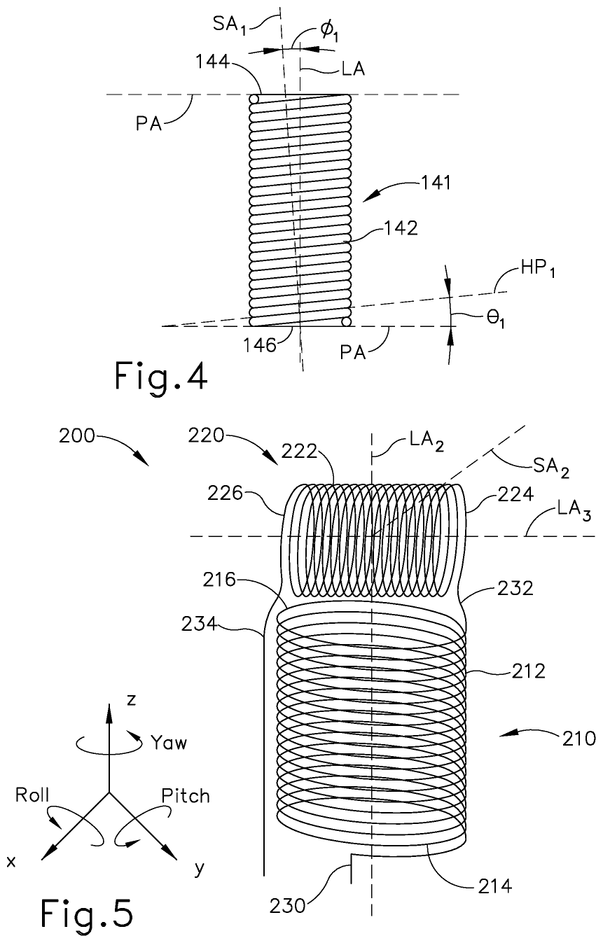

As noted above, sensor (140) of navigation guidewire (130) may take the form of a coil. FIG. 4 shows sensor (140) in the form of an exemplary coil (141). In this example, coil (141) is formed by a single, electrically conductive wire (142) that is wound in helical configuration. In other words, coil (141) is formed from a plurality of wire loops that are tightly stacked on top of one another and all are part of the same conductive wire (142). The helical configuration of coil (141) is longitudinally compressed with a fine pitch, in that each winding of wire (142) is in contact with the adjacent windings of wire (142). Coil (141) is centered along a central longitudinal axis (LA₁). In some versions, this central longitudinal axis (LA₁) is aligned with the central longitudinal axis of navigation guidewire (130). In the present example, the plane of each loop or winding is substantially perpendicular to longitudinal axis (LA₁) and the center of each loop or winding falls on the longitudinal axis (LA₁). Each terminal end winding (144, 146) of coil (141) generally extends along a respective plane that is perpendicular to longitudinal axis (LA₁) in this example.

FIG. 4 also shows a helix plane (HP), which is defined as the plane indicating the orientation of each helical winding of wire (142). In other words, each winding is oriented along a respective helix plane (HP). A sensor axis (SA₁) extends perpendicularly from helix plane (HP). Sensor axis (SA₁) defines a sensor angle (pi) with longitudinal axis (LA₁). Sensor angle (p) is relatively small (e.g., less than approximately 1°) in this example.

By way of example only, coil (141) may be formed by wrapping wire (142) about a mandrel (not shown). The mandrel may comprise a cylindrical body that has a longitudinal axis aligned with longitudinal axis (LA₁) of the ultimately formed coil (141). Wire (142) is then wrapped around a winding axis that is parallel with the sensor axis (SA₁) of the ultimately formed coil (141). Since the sensor angle (φ₁) is relatively small, the winding axis is substantially aligned with the longitudinal axis of the mandrel.

As noted above, positioning of coil (141) within an alternating current electromagnetic field generated by field generators (206) will induce an electrical current in coil (141); and processor (110) may receive and process this electrical current to determine the location of the distal end (134) of navigation guidewire (130) within a three-dimensional space (i.e., within the head (H) of the patient (P)). In the context of a cartesian coordinate system, processor (110) may determine the x, y, and z coordinates of distal end (134) of guidewire (130) relative to a reference origin. However, with sensor angle (φ₁) being relatively small, it may be very difficult if not impossible for processor (110) to adequately determine the orientation of distal end (134) within three-dimensional space about one of three axes. In particular, processor (110) may be able to adequately determine the roll and pitch orientation of distal end (134) about two respective axes (e.g., x and y) that are perpendicular to the sensor axis (SA₁) or the longitudinal axis (LA₁); but not the yaw orientation of distal end (134) about a third axis (e.g., z) that is substantially aligned with the sensor axis (SA₁) or the longitudinal axis (LA₁). Thus, as coil (141) is rotated about the longitudinal axis (LA₁) in a yaw movement, this yaw movement may induce a virtually imperceptible change in electrical current in coil (141), such that processor (110) may be unable to effectively detect the yaw movement, due to the relatively small sensor angle (φ₁), even if processor (110) is able to detect pitch and roll movements of coil (141).

It may be useful for the operator to know the orientation of distal end (134), in addition to knowing the position of distal end (134), in three-dimensional space. This orientation information may be particularly useful where distal end (134) of guidewire (130) includes a preformed bend (with sensor (140) being distal to the preformed bend). A person skilled in the art may be led to include one or more additional coils in sensor (140) to provide orientation sensing capabilities to sensor (140). In particular, a person skilled in the art may be led to construct an orientation sensitive sensor (140) that includes a first coil configured and oriented like coil (141); and a second coil that is configured like coil (141) but oriented along a different longitudinal axis (LAₙ) that is non-parallel with the longitudinal axis (LA₁) of the first coil (141). This additional coil may enable processor (110) to determine the orientation of the portion of the instrument in which the coils are integrated, in addition to being able to determine the position of the portion of the instrument in which the coils are integrated. However, such an approach may be undesirable to the extent that separate sensor (140) circuits, with separate wire pairs for each sensor (140) are created and coupled with processor (110), as these separate circuits may add to the cost and complexity of the manufacturing process.

Instead of including additional sensor (140) circuits, a person skilled in the art may also be led to include versions of sensor (140) with a sensor axis (SA) that is substantially oblique to the longitudinal axis (LA). Alternatively, a person skilled in the art may also be led to include versions of sensor (140) with a sensor axis (SA) that is curved rather than being straight. To the extent that such variations may provide orientation sensitivity to the sensor (140) (e.g., enabling the sensor (140) to readily provide signals reliably indicating the roll/pitch/yaw angles of distal end (134) about the longitudinal axis (LA)), such variations may again add to the cost and complexity of the manufacturing process.

It may therefore be desirable to provide a version of sensor (140) that provides the enhanced orientation sensitivity that a multi-coil sensor arrangement may provide; while avoiding the potential drawbacks of complexity imposed by a multi-coil sensor arrangement where each coil has its own separate wire pair coupled with processor (110). In particular, it may be desirable to provide a version of sensor (140) that provides the enhanced orientation sensitivity that a conventional multi-coil sensor arrangement may provide, yet only using a single wire pair to couple all of the coils with processor (110).

FIG. 5 shows an exemplary alternative coil assembly (200) that may be incorporated into sensor (140) in place of coil (141) in order to provide enhanced orientation sensitivity to sensor (140). Coil assembly (200) of this example comprises a cascading sensor arrangement formed by a first coil winding (210) and a second coil winding (220), which are coupled together in series. First coil winding (210) is formed by a first wire segment (212); while second coil winding (220) is formed by a second wire segment (222). A first wire (230) is coupled to a first end (214) of first wire segment (212). A second wire (232) joins a second end (232) of first wire segment (212) with a first end (224) of second wire segment (222). A third wire (234) is coupled to a second end (226) of second wire segment (222). In some versions, wires (230, 232, 234) and wire segments (212, 222) are all formed as a single, continuous piece of wire. Coil assembly (200) may thus be formed as a single, unitary piece; without requiring the assembly of separately-formed components.

Wires (230, 234) are further coupled with a coupling unit like coupling unit (132) described above, thereby coupling coil assembly (200) with processor (110) of console (116). The present configuration of coil assembly (200) thus provides coupling of the entire coil assembly (200) with just two wires (230, 234).

First coil winding (210) is oriented along a corresponding longitudinal axis (LA₂); while second coil winding (220) is oriented along another corresponding longitudinal axis (LA₃). In the present example, longitudinal axes (LA₂, LA₃) are perpendicular to each other. In some other variations, longitudinal axes (LA₂, LA₃) are obliquely oriented relative to each other. Sensor assembly (200) defines an effective sensor axis (SA₂) that is a composite of longitudinal axes (LA₂, LA₃). In addition to being a function of the relative orientations of longitudinal axes (LA₂, LA₃), the orientation of sensor axis (SA₂) may also be a function of the number of turns and cross-section of each coil winding (210, 220). For instance, if one coil winding (210, 220) has a greater number of turns and/or a larger cross-section of the other coil winding (210, 220), then sensor axis (SA₂) will be oriented closer to the longitudinal axis (LA₂, LA₃) of the coil winding (210, 220) having the greater number of turns and/or a larger cross-section. In versions where coil windings (210, 220) have the same number of turns and the same cross-sectional dimension, sensor axis (SA₂) will be equiangularly positioned between longitudinal axes (LA₂, LA₃).

In versions where sensor assembly (200) is positioned on the distal end of a medical instrument having a longitudinal axis (not shown) that is aligned with or parallel to either of longitudinal axes (LA₂, LA₃), effective sensor axis (SA₂) will be obliquely oriented relative to the longitudinal axis of the distal end of that medical instrument. Due to this relationship between the effective sensor axis (SA₂) and the longitudinal axis of the distal end of the medical instrument to which sensor assembly (200) is secured, sensor assembly (200) will be able to provide electrical signals indicating the position and the orientation of the distal end of the medical instrument. Sensor assembly (200) may thus provide position and orientation data with a level of detail provided by multi-axis sensors, while only needing two wires (230, 234) to communicate the position and orientation indicative signals. Similarly, sensor assembly (200) may provide position and orientation data with a level of detail provided by sensors having coils with obliquely oriented or curved axes, without imposing additional manufacturing costs or concerns associated with coils having obliquely oriented or curved axes.

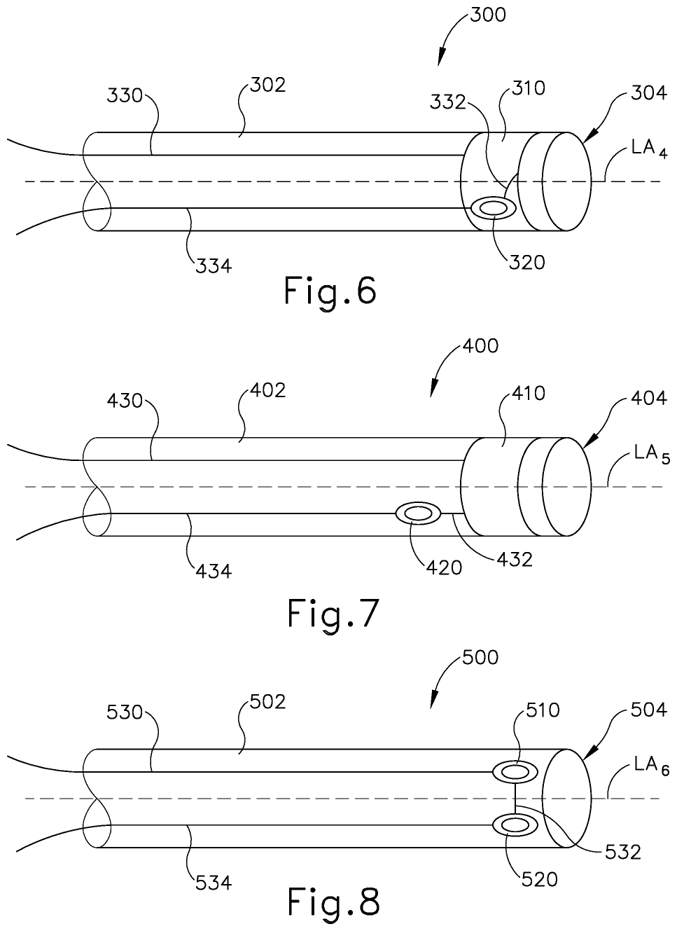

FIG. 6 shows another exemplary sensor assembly coupled to a medical instrument (300). Medical instrument (300) may comprise a guide catheter, a dilation catheter, a guidewire, a suction instrument, a rosen, a curette, a pick, an endoscope, or any other suitable kind of instrument as will be apparent to those skilled in the art in view of the teachings herein. Medical instrument (300) includes a body (302) having a distal end (304) and defining a longitudinal axis (LA₄). A pair of sensors (310, 320) are secured to body (302), near distal end (304). First sensor (310) is in the form of a cylindrical coil that is positioned about the perimeter of body (302), coaxially positioned with longitudinal axis (LA₄). Second sensor (320) is in the form of a pancake coil that is secured to first sensor (310). In some other versions, second sensor (320) is positioned radially between body (302) and first sensor (310). With body (302) having a curved outer profile in the present example, second sensor (320) may be flexible to conform to the curved outer profile of body (302).

In the present example, second sensor (320) is oriented such that an axis passing through the center of second sensor (320) is perpendicular to longitudinal axis (LA₄). Sensors (310, 320) thus cooperate to define an effective sensor axis that is obliquely oriented relative to longitudinal axis (LA₄). Sensors (310, 320) are thus together capable of providing electrical signals indicating the position of distal end (304) in three-dimensional space; and the orientation of distal end (304) about longitudinal axis (LA₄).

Sensors (310, 320) are connected in series in the present example. A first wire (330) is coupled with a first end of first sensor (310), a second wire (332) couples a second end of first sensor (310) with a first end of second sensor (320), and a third wire (334) is coupled with a second end of second sensor (320). Wires (330, 334) are further coupled with a coupling unit like coupling unit (132) described above, thereby coupling sensors (310, 320) with processor (110) of console (116). The configuration shown in FIG. 6 thus provides coupling of two sensors (310, 320) with processor (110) with just two wires (330, 334). The configuration shown in FIG. 6 may thus provide position and orientation data with a level of detail provided by multi-axis sensors, while only needing two wires (330, 334) to communicate the position and orientation indicative signals. Similarly, the configuration shown in FIG. 6 may provide position and orientation data with a level of detail provided by sensors having coils with obliquely oriented or curved axes, without imposing additional manufacturing costs or concerns associated with coils having obliquely oriented or curved axes.

FIG. 7 shows another exemplary sensor assembly coupled to a medical instrument (400). Medical instrument (400) may comprise a guide catheter, a dilation catheter, a guidewire, a suction instrument, a rosen, a curette, a pick, an endoscope, or any other suitable kind of instrument as will be apparent to those skilled in the art in view of the teachings herein. Medical instrument (400) includes a body (402) having a distal end (404) and defining a longitudinal axis (LA₅). A pair of sensors (410, 420) are secured to body (402), near distal end (404). First sensor (410) is in the form of a cylindrical coil that is positioned about the perimeter of body (402), coaxially positioned with longitudinal axis (LA₅). Second sensor (420) is in the form of a pancake coil that is secured to body (402) at a location proximal to first sensor (410). In some other versions, second sensor (420) is positioned distal to first sensor (410). With body (402) having a curved outer profile in the present example, second sensor (420) may be flexible to conform to the curved outer profile of body (402).

In the present example, second sensor (420) is oriented such that an axis passing through the center of second sensor (420) is perpendicular to longitudinal axis (LA₅). Sensors (410, 420) thus cooperate to define an effective sensor axis that is obliquely oriented relative to longitudinal axis (LA₅). Sensors (410, 420) are thus together capable of providing electrical signals indicating the position of distal end (404) in three-dimensional space; and the orientation of distal end (404) about longitudinal axis (LA₅).

Sensors (410, 420) are connected in series in the present example. A first wire (430) is coupled with a first end of first sensor (410), a second wire (432) couples a second end of first sensor (410) with a first end of second sensor (420), and a third wire (434) is coupled with a second end of second sensor (420). Wires (430, 434) are further coupled with a coupling unit like coupling unit (132) described above, thereby coupling sensors (410, 420) with processor (110) of console (116). The configuration shown in FIG. 7 thus provides coupling of two sensors (410, 420) with processor (110) with just two wires (430, 434). The configuration shown in FIG. 7 may thus provide position and orientation data with a level of detail provided by multi-axis sensors, while only needing two wires (430, 434) to communicate the position and orientation indicative signals. Similarly, the configuration shown in FIG. 7 may provide position and orientation data with a level of detail provided by sensors having coils with obliquely oriented or curved axes, without imposing additional manufacturing costs or concerns associated with coils having obliquely oriented or curved axes.

FIG. 8 shows another exemplary sensor assembly coupled to a medical instrument (500). Medical instrument (500) may comprise a guide catheter, a dilation catheter, a guidewire, a suction instrument, a rosen, a curette, a pick, an endoscope, or any other suitable kind of instrument as will be apparent to those skilled in the art in view of the teachings herein. Medical instrument (500) includes a body (502) having a distal end (504) and defining a longitudinal axis (LA₆). A pair of sensors (510, 520) are secured to body (502), near distal end (504). First sensor (510) is in the form of a pancake coil that is secured to body (502). Second sensor (520) is also in the form of a pancake coil that is secured to body (502). In the present example, sensors (510, 520) are located at the same longitudinal position along body (502) but are angularly spaced apart from each other. In some other versions, sensors (510, 520) are located at different longitudinal positions along body (502). In some such versions, sensors (510, 520) are still angularly spaced apart from each other in addition to being longitudinally spaced apart from each other.

With body (502) having a curved outer profile in the present example, each sensor (510, 520) may be flexible to conform to the curved outer profile of body (502). In the present example, each sensor (510, 520) is oriented such that an axis passing through the center of each sensor (510, 520) is perpendicular to longitudinal axis (LA₆). Sensors (510, 520) thus cooperate to define an effective sensor axis that is obliquely oriented relative to longitudinal axis (LA₆). Sensors (510, 520) are thus together capable of providing electrical signals indicating the position of distal end (504) in three-dimensional space; and the orientation of distal end (504) about longitudinal axis (LA₆).

Sensors (510, 520) are connected in series in the present example. A first wire (530) is coupled with a first end of first sensor (510), a second wire (532) couples a second end of first sensor (510) with a first end of second sensor (520), and a third wire (534) is coupled with a second end of second sensor (520). Wires (530, 534) are further coupled with a coupling unit like coupling unit (132) described above, thereby coupling sensors (510, 520) with processor (110) of console (116). The configuration shown in FIG. 8 thus provides coupling of two sensors (510, 520) with processor (110) with just two wires (530, 534). The configuration shown in FIG. 8 may thus provide position and orientation data with a level of detail provided by multi-axis sensors, while only needing two wires (530, 534) to communicate the position and orientation indicative signals. Similarly, the configuration shown in FIG. 8 may provide position and orientation data with a level of detail provided by sensors having coils with obliquely oriented or curved axes, without imposing additional manufacturing costs or concerns associated with coils having obliquely oriented or curved axes.

### IV. EXEMPLARY COMBINATIONS

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus comprising: (a) a medical instrument having a distal end; (b) a sensor assembly positioned at the distal end of the medical instrument, wherein the sensor assembly comprises: (i) a first coil member oriented along a first longitudinal axis, and (ii) a second coil member oriented along a second longitudinal axis, wherein the first and second coil members are coupled with each other in series, wherein the first and second longitudinal axes are offset from each other, wherein the first and second coil members are configured to generate electrical signals indicative of a position and orientation of the distal end of the medical instrument within a patient; (c) a first wire coupled with the sensor assembly; and (d) a second wire coupled with the sensor assembly; wherein the first and second wires are operable to communicate signals from the sensor assembly to a navigation system.

### Example 2

The apparatus of Example 1, wherein the medical instrument is selected from the group consisting of a guide catheter, a dilation catheter, a guidewire, a suction instrument, a rosen, a curette, a pick, and an endoscope.

### Example 3

The apparatus of any one or more of Examples 1 through 2, wherein the distal end is oriented along the first longitudinal axis.

### Example 4

The apparatus of any one or more of Examples 1 through 3, wherein the first and second longitudinal axes are perpendicular to each other.

### Example 5

The apparatus of any one or more of Examples 1 through 4, wherein the sensor assembly defines an effective sensor axis, wherein the effective sensor axis has an orientation based on the orientation of the first longitudinal axis and the orientation of the second longitudinal axis.

### Example 6

The apparatus of Example 5, wherein the first and second coil members each have a respective number of windings, wherein the orientation of the effective sensor axis is further based on a relationship between the number of windings of the first coil member and the number of windings of the second coil member.

### Example 7

The apparatus of any one or more of Examples 5 through 6, wherein the first and second coil members each have a respective cross-sectional size, wherein the orientation of the effective sensor axis is further based on a relationship between the cross-sectional size of the first coil member and the cross-sectional size of the second coil member.

### Example 8

The apparatus of any one or more of Examples 5 through 7, wherein the effective sensor axis is equiangularly spaced from the first and second longitudinal axes.

### Example 9

The apparatus of any one or more of Examples 1 through 8, wherein the first coil member and the second coil member are joined to each other via a joining wire.

### Example 10

The apparatus of Example 9, wherein the first coil member, the joining wire, and the second coil member consist of a single piece of wire.

### Example 11

The apparatus of Example 10, wherein the first wire, the first coil member, the joining member, the second coil member, and the second wire consist of a single piece of wire.

### Example 12

The apparatus of any one or more of Examples 1 through 11, wherein the first coil member comprises a cylindrical coil.

### Example 13

The apparatus of Example 12, wherein the second coil member comprises a pancake coil.

### Example 14

The apparatus of Example 13, wherein the pancake coil is positioned on an exterior of the cylindrical coil.

### Example 15

The apparatus of any one or more of Examples 13 through 14, wherein the pancake coil is longitudinally offset from the cylindrical coil.

### Example 16

The apparatus of any one or more of Examples 1 through 11, wherein the first coil member comprises a first pancake coil, wherein the second coil member comprises a second pancake coil.

### Example 17

The apparatus of Example 16, wherein the first and second pancake coils are positioned at a shared longitudinal position along the medical instrument, wherein the first and second pancake coils are angularly offset from each other.

### Example 18

An apparatus comprising: (a) a medical instrument having a distal end; (b) a sensor assembly positioned at the distal end of the medical instrument, wherein the sensor assembly comprises: (i) a first coil member oriented along a first longitudinal axis, wherein the first coil member has a first end and a second end, (ii) a second coil member oriented along a second longitudinal axis, wherein the second coil member has a first end and a second end, and (iii) a joining wire connecting the second end of the first coil member with the first end of the second coil member, wherein the first and second longitudinal axes are offset from each other, wherein the first and second coil members are configured to generate electrical signals indicative of a position and orientation of the distal end of the medical instrument within a patient, (c) a first wire coupled with the first end of the first coil member; and (d) a second wire coupled with the second end of the second coil member; wherein the first and second wires are operable to communicate signals from the sensor assembly to a navigation system.

### Example 19

The apparatus of Example 18, further comprising an image guided surgery system, wherein the image guided surgery system comprises a processor configured to process signals from the sensor assembly and thereby determine the position and orientation of the distal end of the medical instrument within a patient.

### Example 20

An apparatus comprising: (a) a medical instrument having a distal end; (b) a sensor assembly positioned at the distal end of the medical instrument, wherein the sensor assembly comprises: (i) a first coil member oriented along a first longitudinal axis, and (ii) a second coil member oriented along a second longitudinal axis, wherein the first and second coil members are coupled with each other in series, wherein the first and second longitudinal axes are offset from each other; (c) a first wire coupled with the sensor assembly; (d) a second wire coupled with the sensor assembly; and (e) an image guided surgery system, wherein the image guided surgery system comprises: (i) one or more field generators configured to generate a magnetic field around at least a portion of a patient, wherein the first and second coil members are configured to generate electrical signals, based on the magnetic field generated by the one or more field generators, and (ii) a processor in communication with the first and second wires, wherein the processor is configured to process signals from the sensor assembly received via the first and second wires and thereby determine the position and orientation of the distal end of the medical instrument within a patient

### V. MISCELLANEOUS

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices disclosed herein can be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, versions of the device may be disassembled, and any number of the particular pieces or parts of the device may be selectively replaced or removed in any combination. Upon cleaning and/or replacement of particular parts, versions of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a surgical facility. A device may also be sterilized using any other technique known in the art, including but not limited to beta or gamma radiation, ethylene oxide, or steam.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus comprising:
(a) a medical instrument having a distal end;
(b) a sensor assembly positioned at the distal end of the medical instrument, wherein the sensor assembly comprises:
(i) a first coil member oriented along a first longitudinal axis, and
(ii) a second coil member oriented along a second longitudinal axis,
wherein the first and second coil members are coupled with each other in series,
wherein the first and second longitudinal axes are offset from each other,
wherein the first and second coil members are configured to generate electrical signals indicative of a position and orientation of the distal end of the medical instrument within a patient;
(c) a first wire coupled with the sensor assembly; and
(d) a second wire coupled with the sensor assembly;
wherein the first and second wires are operable to communicate signals from the sensor assembly to a navigation system.

2. The apparatus of claim 1, wherein the medical instrument is selected from the group consisting of a guide catheter, a dilation catheter, a guidewire, a suction instrument, a rosen, a curette, a pick, and an endoscope.

3. The apparatus of claim 1, wherein the distal end is oriented along the first longitudinal axis.

4. The apparatus of claim 1, wherein the first and second longitudinal axes are perpendicular to each other.

5. The apparatus of claim 1, wherein the sensor assembly defines an effective sensor axis, wherein the effective sensor axis has an orientation based on the orientation of the first longitudinal axis and the orientation of the second longitudinal axis.

6. The apparatus of claim 5, wherein the first and second coil members each have a respective number of windings, wherein the orientation of the effective sensor axis is further based on a relationship between the number of windings of the first coil member and the number of windings of the second coil member.

7. The apparatus of claim 5, wherein the first and second coil members each have a respective cross-sectional size, wherein the orientation of the effective sensor axis is further based on a relationship between the cross-sectional size of the first coil member and the cross-sectional size of the second coil member.

8. The apparatus of claim 5, wherein the effective sensor axis is equiangularly spaced from the first and second longitudinal axes.

9. The apparatus of claim 1, wherein the first coil member and the second coil member are joined to each other via a joining wire.

10. The apparatus of claim 9, wherein the first coil member, the joining wire, and the second coil member consist of a single piece of wire.

11. The apparatus of claim 10, wherein the first wire, the first coil member, the joining member, the second coil member, and the second wire consist of a single piece of wire.

12. The apparatus of claim 1, wherein the first coil member comprises a cylindrical coil.

13. The apparatus of claim 12, wherein the second coil member comprises a pancake coil.

14. The apparatus of claim 13, wherein the pancake coil is positioned on an exterior of the cylindrical coil.

15. The apparatus of claim 13, wherein the pancake coil is longitudinally offset from the cylindrical coil.

16. The apparatus of claim 1, wherein the first coil member comprises a first pancake coil, wherein the second coil member comprises a second pancake coil.

17. The apparatus of claim 16, wherein the first and second pancake coils are positioned at a shared longitudinal position along the medical instrument, wherein the first and second pancake coils are angularly offset from each other.

18. An apparatus comprising:
(a) a medical instrument having a distal end;
(b) a sensor assembly positioned at the distal end of the medical instrument, wherein the sensor assembly comprises:
(i) a first coil member oriented along a first longitudinal axis, wherein the first coil member has a first end and a second end,
(ii) a second coil member oriented along a second longitudinal axis, wherein the second coil member has a first end and a second end, and
(iii) a joining wire connecting the second end of the first coil member with the first end of the second coil member,
wherein the first and second longitudinal axes are offset from each other,
wherein the first and second coil members are configured to generate electrical signals indicative of a position and orientation of the distal end of the medical instrument within a patient;
(c) a first wire coupled with the first end of the first coil member; and
(d) a second wire coupled with the second end of the second coil member;
wherein the first and second wires are operable to communicate signals from the sensor assembly to a navigation system.

19. The apparatus of claim 18, further comprising an image guided surgery system, wherein the image guided surgery system comprises a processor configured to process signals from the sensor assembly and thereby determine the position and orientation of the distal end of the medical instrument within a patient.

20. An apparatus comprising:
(a) a medical instrument having a distal end;
(b) a sensor assembly positioned at the distal end of the medical instrument,
wherein the sensor assembly comprises:
(i) a first coil member oriented along a first longitudinal axis, and
(ii) a second coil member oriented along a second longitudinal axis,
wherein the first and second coil members are coupled with each other in series,
wherein the first and second longitudinal axes are offset from each other,
(c) a first wire coupled with the sensor assembly;
(d) a second wire coupled with the sensor assembly; and
(e) an image guided surgery system, wherein the image guided surgery system comprises:
(i) one or more field generators configured to generate a magnetic field around at least a portion of a patient, wherein the first and second coil members are configured to generate electrical signals, based on the magnetic field generated by the one or more field generators, and
(ii) a processor in communication with the first and second wires, wherein the processor is configured to process signals from the sensor assembly received via the first and second wires and thereby determine the position and orientation of the distal end of the medical instrument within a patient.
